(19) United States
(12) Patent Application Publication
(10) Pub. No.: US 2022/0089724 A1
(43) Pub. Date: Mar. 24, 2022

(54) TREATMENT OF NEUROTOXICITY AND/OR CYTOKINE RELEASE SYNDROME

(71) Applicant: AUTOLUS LIMITED, London (GB)

(72) Inventor: Vijay Peddareddigari, London (GB)

(21) Appl. No.: 17/426,443

(22) PCT Filed: Jan. 29, 2020
(86) PCT No.: PCT/GB2020/050208

§ 371 Date: Jul. 28, 2021

(30) Foreign Application Priority Data
Jan. 29, 2019 (GB) 1901187.3

(51) Int. Cl. C07K 16/28; A61K 38/20

(52) U.S. Cl. CPC C07K 16/2803; A61K 38/2006

(57) ABSTRACT

The present invention relates to the use of an IL-1β inhibitor for the prevention and/or treatment of neurotoxicity and/or cytokine release syndrome in a subject undergoing immunotherapy, wherein the IL-1β inhibitor is administered before immunotherapy.

### FIELD OF THE INVENTION

The present invention relates to an IL-1β inhibitor for use in the prevention and/or treatment of neurotoxicity and/or cytokine release syndrome in a subject undergoing immunotherapy, wherein the IL-1β inhibitor is administered before after immunotherapy.

### BACKGROUND TO THE INVENTION

Immunotherapy is a powerful therapeutic tool that stimulates the host immune response to prevent and/or treat disorders. Immunotherapy uses therapeutic factors such as drugs, agents or engineered cells to stimulate the host immune response against target disorder cells, such as for example, cancer cells.

Immunotherapy factors include, but are not limited to, antibody therapies or chimeric antigen receptor-modified T (CAR T) cell therapies. These therapies are typically administered systemically and have shown significant promise in treating disorders, for example many cancers.

However the recent application of immunotherapies in the clinic has raised safety and efficacy concerns in connection with the treatment of some disorders. While immunotherapy harnesses the power of the host immune response, it is known that an uncontrolled inflammatory immune response can result in neurotoxicity and/or Cytokine Release Syndrome (CRS) which can result in a range of symptoms that lead to further illness and death, if left unchecked. For example, anti-leukaemia efficacy by CD19-specific CAR T cells in humans has been associated with neurotoxicity and CRS. It is also known that immunotherapy associated conditions correlate with the activity of a number of cell types and the release of a variety of factors including cytokines.

A large variety of cells and factors regulate the inflammatory immune response to provide protection against pathogenic infection and tumour cells. Cytokines are known to play a key role in the inflammatory immune response and notably, the cytokine IL-6, is known to have pleiotropic roles in regulating immune cell function and inflammatory immune responses.

Therapeutic approaches for the treatment of CRS involve systemic approaches that use anti-IL-6 receptor antibodies or such antibodies with steroids. The anti-IL6 antibody tocilizumab has been used to treat CRS. While neurotoxicity and CRS have well established side-effects associated with immunotherapy, tocilizumab has proven unsuccessful in also treating neurotoxicity.

Accordingly, there remains a need for further approaches to treat and/or prevent neurotoxicity and/or CRS in subjects undergoing immunotherapy.

### SUMMARY OF THE INVENTION

The present inventors have determined that IL-1β inhibitors have utility in preventing and/or treating neurotoxicity and/or CRS by targeting the IL-1β-IL-1 receptor (IL-1R1) axis.

Accordingly in one aspect, the present invention provides an IL-1β inhibitor for use in the prevention and/or treatment of neurotoxicity and/or cytokine release syndrome in a subject undergoing immunotherapy, wherein the IL-1β inhibitor is administered before immunotherapy.

The IL-1β inhibitor may be administered more than once.

The IL-1β inhibitor may be administered about 30 days or less before immunotherapy administration.

The IL-1β inhibitor may be administered 1 to 30 days, 5 to 30 days, 10 to 30 days, 15 to 30 days, 20 to 30 days, or 25 to 30 days before immunotherapy administration.

The IL-1β inhibitor may be administered 1 to 5 days, 5 to 10 days, 10 to 15 days, 15 to 20 days, 20 to 25 days, and/or 25 to 30 days before immunotherapy administration.

The IL-1β inhibitor may be administered 30 days or less, 25 days or less, 20 days or less, 15 days or less, 10 days or less, 5 days or less, 4 days or less, 3 days or less, 2 days or less, or 1 day before immunotherapy administration.

The IL-1β inhibitor may be further administered during immunotherapy administration and/or after immunotherapy administration.

The IL-1β inhibitor may be administered 1 to 30 days, 5 to 30 days, 10 to 30 days, 15 to 30 days, 20 to 30 days, or 25 to 30 days after immunotherapy administration.

The IL-1β inhibitor may be administered 1 to 5 days, 5 to 10 days, 10 to 15 days, 15 to 20 days, 20 to 25 days, and/or 25 to 30 days after immunotherapy administration.

The IL-1β inhibitor may be administered 30 days or less, 25 days or less, 20 days or less, 15 days or less, 10 days or less, 5 days or less, 4 days or less, 3 days or less, 2 days or less, or 1 day after immunotherapy administration.

The IL-1β inhibitor may be administered by subcutaneously, intravenously, parenterally or enterally.

The IL-1β inhibitor may block or sterically hinder the binding of IL-1β to IL-1R1.

The IL-1β inhibitor may be an antibody or antigen-binding fragment thereof, a peptide or a small molecule.

The IL-1β inhibitor may be an anti-IL1β antibody or an antigen-binding fragment thereof. The anti-IL1β antibody may be canakinumab or antigen-binding fragment thereof.

The IL-1β inhibitor may be an IL-1R1 peptide antagonist. The IL-1R1 antagonist may be anakinra.

The subject may be undergoing immunotherapy for cancer.

The immunotherapy may be directly and/or indirectly targeted T-cell mediated immunotherapy.

The immunotherapy may be CAR T cell therapy or transgenic TCR T cell therapy, BiTE therapy or anti-CD3 antibody therapy.

The immunotherapy may be CAR T cell therapy.

The subject may be an adult or a paediatric subject.

In another aspect, the present invention provides a method of prevention and/or treatment of neurotoxicity and/or cytokine release syndrome in a human subject undergoing immunotherapy, the method comprising administering to a human subject in need thereof an IL-1β inhibitor before immunotherapy.

In a further aspect, the present invention provides for the use of an IL-1β inhibitor for the manufacture of a medicament for the prevention and/or treatment of neurotoxicity and/or cytokine release syndrome in a human subject undergoing immunotherapy, wherein the medicament is administered before immunotherapy.

### DETAILED DESCRIPTION OF THE INVENTION

IL-1β Inhibitor

The Interleukin-1 (IL-1β) family consists of 11 cytokines that have known roles in inducing and regulating the inflammatory immune response against pathogenic infections and insults. Interleukin 1β (IL-1β) is a cytokine belonging to the Interleukin 1 family. IL-1β is a pro-inflammatory cytokine (an endogenous pyrogen) that is expressed by a variety of cells, including macrophages, NK cells, monocytes and neutrophils.

Under normal conditions, IL-1β is typically expressed in an inactive form as a cytosolic precursor at low levels. Upon insult by a foreign agent or pathogen which stimulates inflammasome-activation, IL-1β levels increase and IL-1β is cleaved by caspase-1 into its active form which is secreted extracellularly. IL-1β binds to the IL-1 receptor (IL-1R1). Upon IL-1β binding to IL-1R1 a complex is formed with high-affinity which followed by binding of additional complex members and adaptor molecules, activates IL-1β mediated signalling pathways including MAPK and NF-κB. These signalling pathways promote the inflammatory immune response as well as other parts of and the innate and adaptive immune system.

The IL-1R1 receptor does not exclusively bind IL-1β; however the effects of IL-1β on inflammation are exerted through IL-1R1.

An illustrative amino acid sequence of human IL-1β precursor from Uniprot accession P01584 is set forth in SEQ ID NO: 1. Suitably, IL-1β precursor comprises an amino acid sequence as set forth in SEQ ID NO: 1 or a variant thereof.

(SEQ ID NO: 1)
MAEVPELASEMMAYYSGNEDDLFFEADGPKQMKCSFQDLDLCPLDGGIQL
RISDHHYSKGFRQAASVVVAMDKLRKMLVPCPQTFQENDLSTFFPFIFEE
EPIFFDTWDNEAYVHDAPVRSLNCTLRDSQQKSLVMSGPYELKALHLQGQ
DMEQQVVFSMSFVQGEESNDKIPVALGLKEKNLYLSCVLKDDKPTLQLES
VDPKNYPKKKMEKRFVFNKIEINNKLEFESAQFPNWYISTSQAENMPVFL
GGTKGGQDITDFTMQFVSS.

An illustrative amino acid sequence of human IL-1β (after cleavage from the precursor) from Uniprot accession C9JVK0 is set forth in SEQ ID NO: 2. Suitably, IL-1β comprises an amino acid sequence as set forth in SEQ ID NO: 2 or a variant thereof.

(SEQ ID NO: 2)
MAEVPELASEMMAYYSGNEDDLFFEADGPKQMKCSFQDLDLCPLDGGIQL
RISDHHYSKGFRQAASVVVAMDKLRKMLVPCPQTFQENDLSTFFPFIFEE
EPIFFDTWDNE.

An illustrative amino acid sequence of the human IL-1R1 from Uniprot accession P14778 is set forth in SEQ ID NO: 3. Suitably, IL-1R1 comprises an amino acid sequence as set forth in SEQ ID NO: 3 or a variant thereof.

(SEQ ID NO: 3)
MKVLLRLICFIALLISSLEADKCKEREEKIILVSSANEIDVRPCPLNPNE
HKGTITWYKDDSKTPVSTEQASRIHQHKEKLWFVPAKVEDSGHYYCVVRN
SSYCLRIKISAKFVENEPNLCYNAQAIFKQKLPVAGDGGLVCPYMEFFKN
ENNELPKLQWYKDCKPLLLDNIHFSGVKDRLIVMNVAEKHRGNYTCHASY
TYLGKQYPITRVIEFITLEENKPTRPVIVSPANETMEVDLGSQIQLICNV
TGQLSDIAYWKWNGSVIDEDDPVLGEDYYSVENPANKRRSTLITVLNISE
IESRFYKHPFTCFAKNTHGIDAAYIQLIYPVTNFQKHMIGICVTLTVIIV
CSVFIYKIFKIDIVLWYRDSCYDFLPIKASDGKTYDAYILYPKTVGEGST
SDCDIFVFKVLPEVLEKQCGYKLFIYGRDDYVGEDIVEVINENVKKSRRL
IIILVRETSGFSWLGGSSEEQIAMYNALVQDGIKVVLLELEKIQDYEKMP
ESIKFIKQKHGAIRWSGDFTQGPQSAKTRFWKNVRYHMPVQRRSPSSKHQ
LLSPATKEKLQREAHVPLG.

As defined herein, an “IL-1β inhibitor” is an entity that reduces or inhibits the biological activity of IL-1β. In other words, the IL-1β induction of a biological signaling response is reduced or inhibited. In one embodiment, an IL-1β inhibitor of the present invention reduces or inhibits IL-1β hyperactivation signalling for the inflammatory immune response.

The term “inhibitor” or “inhibition” refers to the reduction or prevention of IL-1R1 signalling that results from IL-1β binding to IL-1R1. Suitably, the inhibitor or inhibition targets and disrupts the IL-1β-IL-1R1 axis. This disruption reduces the signalling activity of IL-1β. As will be apparent, the inhibition/reduction in activity refers to a comparison to the activity in a corresponding control cell which is treated in identical conditions apart from the control cell is not treated with the IL-1β inhibitor.

Suitable assays for determining IL-1β inhibition are known in the art and include, for example, reporter cell lines and measuring IL-1β levels, IL-1R1 levels or IL-1β mediated signalling pathways by various means including, for example, ELISA. The ability of IL-1β to specifically bind IL-1R1 may be determined using methods which are known in the art. For example, determination of binding affinity (e.g. using a BIAcore instrument), western blot, flow cytometry, in situ hybridisation and/or microscopy. In one embodiment, a competitive binding assay for IL-1β with IL-1R1 may be applied. Alternatively any assay that quantitatively assesses downstream signalling may be used.

Suitably, the IL-1β inhibitor is capable of specifically binding a target polypeptide in order to inhibit the activity of IL-1β transduction and, accordingly, modulate the IL-1β-IL-1R1 axis.

“Modulate” may refer to decreasing, reducing or eliminating the signalling activity of IL-1β through IL-1R1, in other words IL-1β signal transduction.

A “target polypeptide” may refer to IL-1β, IL-1R1 or any polypeptide that mediates signalling via the IL-1β-IL-1R1 axis. Examples of other polypeptides that inhibit the IL-1β-IL-1R1 receptor axis include known accessory proteins for the IL-1β-IL1R1 bound complex, or Interleukin-1 receptor accessory protein (ILRAP), which is an IL-1β-IL1R1 complex co-receptor known to be required for IL-1β transduction once IL-1β is bound to IL-1R1.

Suitably, the target polypeptide may be IL-1β, IL-1R1 or ILRAP.

Decreasing IL-1β activity may mean a reduction/inhibition in the signalling activity mediated by IL-1β binding to IL-1R1 by at least 10%, at least 20%, at least 30%, at least 40%, at least 50%, at least 60%, at least 70%, at least 80%, at least 90%, at least 95% or at least 99%. As will be apparent, the decreased activity may be in comparison to the level of IL-1β activity in the absence of the IL-1β inhibitor. Suitably, the signalling activity mediated by IL-1β binding to IL-1R1 may be eliminated.

IL-1β/IL-1R1

According to the present invention, the IL-1β inhibitor may target IL-1β and/or IL-1R1 directly or indirectly. Suitably, the IL-1β inhibitor may target IL-1β indirectly by targeting ILRAP or an IL-1β/IL-1R1 complex accessory protein.

Suitable IL-1β inhibitors are known in the art. Such inhibitors may bind soluble IL-1β and block its binding to IL-1R1. This may be achieved by the inhibitor acting to reduce the free levels of IL-1β and/or blocking its binding site with IL-1R1. In addition, IL-1β inhibitors that are IL-1R1 antagonists are known in the art.

As used herein, the term “IL-1R1 antagonist” is defined as an agent that binds to IL-1R1 either directly or indirectly and reduces or prevents IL-1R1 signal transduction associated with the binding of IL-1β to IL-1R1, such that the transduction of biological activity of IL-1β is reduced or inhibited. Suitably, the IL-1R1 antagonist binds directed to IL-R1 and reduces or prevents IL-1R1 signal transduction associated with the binding of IL-1β to IL-1R1.

Suitably, the IL-1R1 antagonist is an IL-1R1 peptide antagonist.

In one embodiment, the IL-1β inhibitor is an IL-1R1 antagonist that is targeted towards IL-1R1. Suitably, the antagonist may directly bind to IL-1R1 or may reduce or inhibit IL-1β signalling via IL-1R1 through an indirect effect on IL-1R1, by binding for example to ILRAP.

In one embodiment, the IL-1β inhibitor reduces or eliminates an interaction between IL-1β and IL-1R1. Suitably, the protein-protein interaction of IL-1β and IL-1R1 may be disrupted in the presence of the IL-1β inhibitor i.e. the inhibitor acts as a “disruptor” of binding between IL-1β and IL-1R1.

Suitably, binding between IL-1β and IL-1R1 may be reduced by at least 10%, at least 20%, at least 30%, at least 40%, at least 50%, at least 60%, at least 70%, at least 80%, at least 90%, at least 95% or at least 99% by the IL-1β inhibitor. Suitably, binding between IL-1β and IL-1R1 may be eliminated.

Suitably, the IL-1β inhibitor may be capable of specifically binding the target polypeptide at a higher affinity than the binding between the target polypeptide and the second polypeptide.

As used herein, the “target polypeptide” and “second polypeptide” refers to two or more polypeptides that bind each other directly or indirectly to mediate IL-1β signal transduction. For example, any one of the “target polypeptide” or “second polypeptide” may be IL-1β, IL-1R1, ILRAP or an alternative protein that alters binding between IL-1β and IL-1R1 or alters the binding of cofactors or accessory proteins that inhibits IL-1β transduction upon binding to IL-1R1.

Suitably, the target polypeptide may be IL-1β and the second polypeptide may be IL-1R1. Suitably, the target polypeptide may be IL-1R1 and the second polypeptide may be IL-1β. Suitably, the target polypeptide may be ILRAP and the second polypeptide may be IL-1R1.

As used herein, “higher affinity” means that the IL-1β inhibitor binds to the target polypeptide with at least 5, 10, 20, 50, 100, 1000 or 10000-fold greater affinity than the binding affinity between the target polypeptide and the second polypeptide.

Assays for measuring binding affinity and competitive binding are known in the art such as radioactive ligand binding assays (including saturation binding, Scatchard plot), non-radioactive ligand binding assays (including fluorescence polarization, fluorescence resonance energy transfer and surface plasmon resonance/Biacore, and solid phase ligand binding assays.

The IL-1β inhibitor may reduce/inhibit the interaction between the IL-1β and IL-1R1 by, for example, competitive binding to, or steric hindrance of, the target polypeptide, preferably wherein the target polypeptide is IL-1β or IL-1R1.

Suitably, the interaction between the IL-1β and IL-1R1 interaction domain may be disrupted by the IL-1β inhibitor binding competitively to IL-1β or IL-1R1.

Suitably, the IL-1β inhibitor may bind competitively by directly binding to a binding site on the target polypeptide which interacts with the reciprocal binding site on the second polypeptide. Alternatively, the IL-1β inhibitor may bind competitively by binding to a region which overlaps with the binding site on the target polypeptide which interacts with the reciprocal binding site on the second polypeptide.

In one embodiment, binding of the IL-1β inhibitor to its target polypeptide at a region that results in competitive binding of the target polypeptide may also be referred to herein as “blocking” of the target polypeptide.

Suitably, the IL-1β inhibitor may inhibit an interaction between the IL-1β and IL-1R1 via a steric hindrance.

In one embodiment, the IL-1β inhibitor of the present invention blocks or sterically hinders binding of IL-1β to IL-1R1.

As used herein, “blocks” refers to the reduction or elimination of IL-1β binding to IL-1R1.

As used herein, “steric hindrance” refers to the IL-1β inhibitor binding to the target polypeptide at a site that is distinct from the binding site that facilitates the interaction between IL-1β and the IL-1R1; wherein said binding between the IL-1β inhibitor and the target polypeptide reduces/inhibits a binding interaction between IL-1β and IL-1R1. For example, binding between IL-1β inhibitor and the target polypeptide may induce a conformational shift in the target polypeptide that reduces/inhibits binding between IL-1β and IL-1R1.

Non-limiting examples of IL-1β inhibitor mechanisms for mediating inhibition of IL-1β are described herein and additional mechanisms are well known in the art.

IL-1β Inhibitor

In one embodiment, the IL-1β inhibitor may comprise an antibody or antigen-binding fragment thereof.

Suitably, the antibody or antigen-binding fragment thereof may be a full-length antibody, a single chain antibody fragment, a F(ab) fragment, a F(ab′)2 fragment, a F(ab′) fragment, a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, a domain antibody (DAb), an abdurin/nanoantibody, a centyrin, an alphabody or a nanofitin which is capable of binding a IL-1β or IL-1R1 inhibiting IL-1β transduction.

IL-1β inhibitor antibodies are well known in the art.

Suitably, the IL-1β inhibitor antibody may be a blocking or an antagonistic antibody.

In one embodiment, the IL-1β inhibitor is an IL-1β blocking antibody or antigen-binding fragment thereof. IL-1β blocking antibodies include but are not limited to canakinumab, gevokizumab, LY2189102 and MABp1.

Canakinumab is a human monoclonal antibody that functions by binding to IL-1β and preventing it from binding to IL-1R1, therefore inhibiting IL-1β signal transduction. Canakinumab has been shown to reduce IL-1β mediated inflammation for the treatment of rheumatoid arthritis and Cryopyrin-Associated Periodic Syndrome (CAPS). Canakinumab has the approved trade name Ilaris® and is disclosed in WO 02/16436.

In one embodiment, the IL-1β inhibitor may comprise a non-antibody polypeptide or a variant thereof.

The term “non-antibody polypeptide” refers to a binding polypeptide that does not bind to its target polypeptide via complementary determining regions (CDRs). The non-antibody polypeptide may also be referred to as a polypeptide.

The terms “protein”, “polypeptide” and “peptide” are used synonymously herein.

In one embodiment, the IL-1β inhibitor may comprise a fusion polypeptide.

Suitably, the IL-1β inhibitor may comprise a soluble receptor which traps the target IL-1β. The term “traps” refers to an inhibitor that binds to IL-1β either directly or indirectly and functionally and/or physically sequesters IL-1β such that its signalling transduction is inhibited.

Suitably, the IL-1β inhibitor may comprise a non-polypeptide molecule. Suitably, the IL-1β inhibitor may comprise a small molecule.

In one embodiment, the IL-1β inhibitor comprises an IL-1R1 receptor antagonist or a functional derivative, analogue or variant thereof. IL-1R1 receptor antagonists include but are not limited to anakinra or MEDI-8968. In one embodiment, the IL-1β inhibitor is anakinra or a functional derivative or analogue thereof.

Anakinra is an IL-1R1 antagonist that is a modified form of the Interleukin-1 receptor antagonist protein (IL1RA). An illustrative amino acid sequence of 11_1 RA from Uniprot accession P18510 is set forth in SEQ ID NO: 4. Suitably, the IL-1β inhibitor may comprise an amino acid sequence as set forth in SEQ ID NO: 4 or a variant thereof.

(SEQ ID NO: 4)
MEICRGLRSHLITLLLFLFHSETICRPSGRKSSKMQAFRIWDVNQKTFYL
RNNQLVAGYLQGPNVNLEEKIDVVPIEPHALFLGIHGGKMCLSCVKSGDE
TRLQLEAVNITDLSENRKQDKRFAFIRSDSGPTTSFESAACPGWFLCTAM
EADQPVSLTNMPDEGVMVTKFYFQEDE.

IL1RA is known to inhibit IL-1R1 binding to the co-receptor IL1RAP, inhibiting downstream signalling of the IL-1β-IL-1R1 signalling axis. Anakinra is known to have a 152 amino acid sequence shown as positions 26-177 in NCO Reference Sequence NP_776214.1 (www.ncbi.nlm.nih.gov). Herein the term “anakinra or a functional derivative or analogue thereof” comprises amino acid variants having at least 90%, 95%, 96%, 97%, 98% or 99% identity with SEQ ID NO: 4 (the anakinra amino acid sequence).

In one embodiment, the IL-1β inhibitor comprises rilonacept, CYT013, CMPX-1023 or VX-765. In one embodiment, the IL-1β inhibitor comprises rilonacept or a variant thereof.

SEQ ID NO: 5 (illustrative sequence
for rilonacept)
(SEQ ID NO: XX)
SERCDDWGLDTMRQIQVFEDEPARIKCPLFEHFLKFNYSTAHSAGLTLIW
YWTRQDRDLEEPINFRLPENRISKEKDVLWFRPTLLNDTGNYTCMLRNTT
YCSKVAFPLEVVQKDSCFNSPMKLPVHKLYIEYGIQRITCPNVDGYFPSS
VKPTITWYMGCYKIQNFNNVIPEGMNLSFLIALISNNGNYTCVVTYPENG
RTFHLTRTLTVKVVGSPKNAVPPVIHSPNDHVVYEKEPGEELLIPCTVYF
SFLMDSRNEVWWTIDGKKPDDITIDVTINESISHSRTEDETRTQILSIKK
VTSEDLKRSYVCHARSAKGEVAKAAKVKQKVPAPRYTVEKCKEREEKIIL
VSSANEIDVRPCPLNPNEHKGTITWYKDDSKTPVSTEQASRIHQHKEKLW
FVPAKVEDSGHYYCVVRNSSYCLRIKISAKFVENEPNLCYNAQAIFKQKL
PVAGDGGLVCPYMEFFKNENNELPKLQWYKDCKPLLLDNIHFSGVKDRLI
VMNVAEKHRGNYTCHASYTYLGKQYPITRVIEFITLEENKPTRPVIVSPA
NETMEVDLGSQIQLICNVTGQLSDIAYWKWNGSVIDEDDPVLGEDYYSVE
NPANKRRSTLITVLNISEIESRFYKHPFTCFAKNTHGIDAAYIQLIYPVT
NSGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

The IL-1β inhibitor may be a polypeptide comprising SEQ ID NO: 5 or a variant having at least 90%, 95%, 96%, 97%, 98% or 99% identity to SEQ ID NO: 5.

Rilonacept is a soluble recombinant protein that inhibits IL-1β by functioning as an IL-1β trap. Rilonacept is a dimeric fusion protein consisting of portions of IL-1R1 and the IL-1R1 accessory protein linked to the Fc portion of immunoglobulin G1. Rilonacept has high binding affinity towards IL-1β. Rilonacept has the approved trade name Arcalyst®.

The term “small molecule” refers to a low molecular weight organic compound and is well known in the art.

By way of non-limiting example, the IL-1β inhibitor may comprise an anti-IL-1β antibody, an anti-IL-1R1 antibody, an altered IL-1R1 receptor or a partial peptide of IL-1β. The IL-1β inhibitor may be an IL-1R1 receptor antagonist, a decoy receptor, a trap, a dominant-negative receptor or a negative regulator.

IL-1β inhibitors for use in the present invention may be of any origin, any kind, and any form, as long as they exhibit a preventative and/or therapeutic effect on neurotoxicity and/or CRS by inhibiting IL-1β transduction. Accordingly, derivatives, analogs and variants of the IL-1β inhibitors are envisaged herein.

A “derivative” or “analogue” as defined herein is a generic term for a modified protein or small molecule comprising a sequence or structure homologous with a native sequence or structure, that retains the function of the homologous sequence or structure. In connection with a protein, it is understood that many combinations of deletions, insertions, inversions and substitutions can be made to a native amino acid sequence. In connection with a small molecule, it is also understood that many combinations of modifications can be made to a small molecule structure.

A “variant” as defined herein is a generic term for any substitution of, variation of, modification of, replacement of, deletion of or addition of one (or more) amino acids from a polypeptide sequence.

A variant polypeptide according to the present invention may have, for example, one, two or three or more amino acid mutations, for example one, two or three or more amino acid substitutions with respect to the amino acid sequences of polypeptides disclosed herein. Preferably, the amino acid substitutions are conservative substitutions.

As used herein, a variant sequence may have at least 80%, at least 85%, at least 90%, at least 95%, at least 97%, or at least 99% sequence identity to a corresponding reference sequence.

The percentage identity between two sequences may be readily determined by programs such as BLAST, which is freely available at http://blast.ncbi.nlm.nih.gov. Suitably, the percentage identity is determined across the entirety of the reference and/or the query sequence.

Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses homologous substitution (substitution and replacement are both used herein to mean the interchange of an existing amino acid residue, with an alternative residue) i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below. An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue.

Suitably, variant IL-1β inhibitors maintain their ability to inhibit IL-1β transduction.

Suitably, variant IL-1β inhibitors block the binding of wild-type IL-1β to wild-type IL-1R1.

Suitably, variant IL-1β inhibitors may inhibit IL-1β biological transduction through targeting IL-1R1.

### Administration

The present invention is directed to preventing and/or treating neurotoxicity and/or CRS, wherein an IL-1β inhibitor is administered to a subject before the subject administration of an immunotherapy treatment.

The term “undergoing immunotherapy” is defined as encompassing a subject who has been selected to undergo immunotherapy in the future but is yet to begin immunotherapy, and/or a subject concurrently undergoing immunotherapy and/or a subject who has undergone immunotherapy. It is understood that immunotherapy encompasses the administration of the therapeutic to the patient, either directly or indirectly.

According to the present invention, the IL-1β inhibitor is administered before immunotherapy. In one embodiment, the IL-1β inhibitor is further administered during or concurrently with immunotherapy. In one embodiment, the IL-1β inhibitor is administered after immunotherapy.

The term “before immunotherapy” may refer to any point in time after a subject has been selected to undergo immunotherapy but prior to the commencement of administration of the immunotherapy treatment.

According to the present invention, the IL-1β inhibitor may be administered about 30 days or less before immunotherapy administration. The IL-1β inhibitor may be administered 1 to 30 days, 5 to 30 days, 10 to 30 days, 15 to 30 days, 20 to 30 days, or 25 to 30 days before immunotherapy administration. The IL-1β inhibitor may be administered 1 to 5 days, 5 to 10 days, 10 to 15 days, 15 to 20 days, 20 to 25 days, and/or 25 to 30 days before immunotherapy administration The IL-1β inhibitor may be administered 30 days or less, 25 days or less, 20 days or less, 15 days or less, 10 days or less, 5 days or less, 4 days or less, 3 days or less, 2 days or less, or 1 day before immunotherapy administration.

The term “during immunotherapy” may refer to any point in time while the immunotherapy is being administered through an individual treatment or treatment programme over time, or concurrently as the immunotherapy is administered.

Suitably, the IL-1β inhibitor and immunotherapy may be administered concurrently.

As used herein, “concurrent” is used to mean administration of the IL-1β inhibitor and immunotherapy by simultaneous, sequential or separate means.

As used herein, “simultaneous” is used to mean that the IL-1β inhibitor and immunotherapy are administered at an overlapping or the same time. As used herein, “sequential” is used to mean that the IL-1β inhibitor and immunotherapy are not administered concurrently, but one after the other. In contrast to “sequentially”, “separately” is used herein to mean that the gap between administering one agent and the other is significant i.e. the first administered agent may no longer be present in the bloodstream in a therapeutically effective amount when the second agent is administered.

Thus, administration “sequentially” may permit the IL-1β inhibitor to be administered within 5 minutes, 10 minutes or a matter of hours before or after the immunotherapy administration provided the circulatory half-life of the first administered agent is such that they are both concurrently present in therapeutically effective amounts. The time delay between administration of the IL-1β inhibitor and immunotherapy will vary depending on the exact nature of the IL-1β inhibitor and immunotherapy, the interaction there between, and their respective half-lives.

The term “after immunotherapy” may refer to any point in time after the administration of any individual immunotherapy treatment or treatment programme has completed.

According to the present invention, the IL-1β inhibitor may be administered about 30 days or more after immunotherapy administration. The IL-1β inhibitor may be administered 1 to 30 days, 5 to 30 days, 10 to 30 days, 15 to 30 days, 20 to 30 days, or 25 to 30 days after immunotherapy administration. The IL-1β inhibitor may be administered 1 to 5 days, 5 to 10 days, 10 to 15 days, 15 to 20 days, 20 to 25 days, and/or 25 to 30 days after immunotherapy administration. The IL-1β inhibitor may be administered 30 days or less, 25 days or less, 20 days or less, 15 days or less, 10 days or less, 5 days or less, 4 days or less, 3 days or less, 2 days or less, or 1 day after immunotherapy administration.

While the underlying affects of the immunotherapy that has been administered may be ongoing in the subject, “after” as defined herein, relates to the timing of administration of IL-1β inhibitor after the final immunotherapy administration event.

In one embodiment, the IL-1β inhibitor according to the present invention may be administered once. In another embodiment the IL-1β inhibitor may be administered more than once, for example two, three, four, five, six, seven, eight, nine, ten or more times.

Immunotherapy

In accordance with the present invention, “immunotherapy” is defined herein as any therapeutic approach that prevents or treats a disorder by stimulating or modifying the immune response. According to the invention, the immunotherapy may result in neurotoxicity and/or CRS. Suitably, immunotherapy can relate to modifying any part of the immune system. In one embodiment, immunotherapy relates to the induction of the inflammatory response. The immunotherapy may be used to prevent and/or treat any appropriate disorder. Examples of disorders that may be prevented and/or treated by immunotherapy include but are not limited to, cancer, Crohn's disease or rheumatoid arthritis.

Immunotherapies have been shown to be highly effective in treating disorders. For example CAR T cell therapy is known to be highly effective in preventing and/or treating acute lymphoblastic leukaemia (ALL).

Suitably, the subject may be undergoing immunotherapy for any disorder associated with the immune system either directly or indirectly. In one embodiment, the disorder that is being prevented and/or treated by immunotherapy is cancer. Examples of cancer include but are not limited to leukaemia, lymphoma, solid tumours that metastasize to brain, for example, breast, renal and brain cancers. Suitably, the cancer may be a solid tumour. Suitably the cancer may be a cancer such as neuroblastoma, prostate cancer, bladder cancer, breast cancer, colon cancer, endometrial cancer, kidney cancer (renal cell), leukaemia, lung cancer, melanoma, non-Hodgkin lymphoma, pancreatic cancer, and thyroid cancer.

Various tumour associated antigens (TAA) are known, as shown in the following Table 1. The immunotherapy used in the present invention may be targeted against a TAA as indicated therein.

Suitably, the immunotherapy may be a CAR therapy, in particular a CAR T cell therapy, which is targeted against a TAA as indicated in Table 1.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Suitably, the immunotherapy may be an antibody therapy, preferably a monoclonal antibody therapy. Suitably, the antibody therapy may comprise use of antibody fragments. Suitably, the antibody therapy may target CD3 and a target antigen. Suitably, the immunotherapy may be an anti-CD3 antibody therapy.

Suitably, the immunotherapy may be a non-specific therapy, preferably an interferon, interleukin, cytokine or chemokine therapy.

Suitably, the immunotherapy may be a vaccine therapy or virus therapy, preferably an oncolytic virus therapy.

Suitably, the immunotherapy may be a cell therapy or engineered cell therapy. Suitably, the immunotherapy may be a T-cell therapy, preferably a CAR T cell therapy or a transgenic TCR T cell therapy. Suitably, the immunotherapy may be a BiTE therapy.

Immunotherapies comprising cell therapies are well known in the art. For example, in cancer treatment, CAR T cell therapy is known to obtain T cells from a patient and modify the cells to express a chimeric antigen receptor. Said cells are cultured and infused back into the patient for immunotherapy. Many variations and alternative approaches are also well known in the art.

The method of cell-mediated immunotherapy, for example CAR T cell immunotherapy, may involve the steps of:

(i) isolating a cell-containing sample;
(ii) introducing a nucleic acid construct encoding a CAR to the cell; and
(iii) administering the cells from (ii) to a subject.

Suitably, the nucleic acid construct, vector(s) or nucleic acids may be introduced by transduction. Suitably, the nucleic acid construct, vector(s) or nucleic acids may be introduced by transfection.

Suitably, the cell may be autologous. Suitably, the cell may be allogenic.

The engineered immune effector cell may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Suitably, the present the disorder may be targeted by immunotherapy directly and/or indirectly.

Neurotoxicty and/or CRS

The present invention is directed to preventing and/or treating neurotoxicity and/or CRS, preferably wherein the neurotoxicity and/or CRS arise due to immunotherapy.

Neurotoxicity and/or CRS are well known to be associated with immunotherapies. For example, CRS is known to develop in response to monoclonal antibody therapies (such as with OKT-3 and Rituximab) or T-cell therapies, such as CAR T cell therapy targeted against CD19 to treat ALL.

In one embodiment, the neurotoxicity and/or CRS may be the result of administered engineered cells inducing host immune cell activation. In a further embodiment, the neurotoxicity and/or CRS may be the result of administered cells inducing tumour microenvironment cell activation. Alternatively, the neurotoxicity and/or CRS may be the result of tumour microenvironment cells.

Neurotoxicity

In one embodiment, the invention provides for the prevention and/or treatment of neurotoxicity, preferably wherein the neurotoxicity is immune induced neurotoxicity. Suitably, the neurotoxicity may be the result of immunotherapy and/or a disorder.

Immunotherapy related neurotoxicity is well known in the art. Neurotoxicity may include cerebellar syndrome—acute (confusion), cerebral edema (swelling of the brain), cerebral hemorrhage (bleeding of the brain), cerebral herniation, cerebral infarction (stroke), cerebral ischemia (stroke), cerebrovascular accident (stroke) and/or cerebrospinal fluid leak (from the brain or spinal cord), meningeal inflammation and neurodegeneration.

Neurotoxicity may result in a broad range of symptoms. In the case of immunotherapy induced neurotoxicity, the symptoms depend on the response of the subject to immunotherapy. Measurements for the severity of neurotoxicity symptoms are well known in the art. For example, the Common Terminology Criteria for Adverse Events (CTCAE) scale for cancer treatment patients measure the severity of symptoms (including neurotoxicity symptoms) as: 1, mild; 2, moderate; 3, severe; 4, life-threatening or disabling; 5, fatal.

By way of non limiting example, use of the bispecific antibody blinatumomab which targets CD3/CD19 to treat Acute Lymphoblastic Leukemia (ALL) is known to be associated with neurotoxicity including convulsions. CAR T cell therapy targeted against CD19 to treat ALL is known to be associated with tremor, encelopathy, cerebellar alteration, or seizures. A close relationship for neurotoxicity with the inflammatory cytokines associated with CRS is also known in the art.

The symptoms of neurotoxicity place a significant burden on hospitals and carers for monitoring and treatment. Neurotoxicity can result in long term damage to the central nervous system requiring long term care. Neurotoxicity can also lead to death. The present invention advantageously provides a means for the treatment and/or prevention of complex symptoms and conditions that result from neurotoxicity, improving patient health and the time and resources of hospitals and carers.

Advantageously, the present invention provides a means for preventing and/or treating neurotoxicity. Suitably, the present invention may reduce or eliminate one or more of the known neurotoxicity symptoms or side-effects of the subject.

Cytokine Release Syndrome (CRS)

CRS is known to occur as a result of hyper activation of the immune system and is well characterised in the art. CRS is known to clinically manifest when large numbers of lymphocytes and/or myeloid cells release inflammatory cytokines upon activation.

CRS symptom onset may arise within hours or days of immunotherapy, for example with respect to when a patient undergoes immune cell infusion. Symptom onset depends on the degree of immune cell activation. CRS symptoms include but are not limited to, fever, rigors, fatigue, anorexia, myalgias, arthalgias, nausea, vomiting, headache, rash, diarrhoea, tachypnea, hypoxemia, tachycardia, hypotension, widened pulse pressure, early increased cardiac output, late diminished cardiac output, hallucinations, tremor, altered gait, seizures and death.

The present invention is directed to preventing and/or treating CRS. The symptoms of CRS place a significant burden on hospitals and carers for monitoring and treatment. For example, CRS is associated with a very high fever which itself can result in significant complications and lead to death. CRS is associated with rapid onset cardiac dysfunction even though with timely treatment this can be reversible. Hence the present invention provides for the treatment and/or prevention of complex conditions that result from CRS, for example by reducing or eliminating very high fever, improving patient health and the time and resources of hospitals and carers.

Neurotoxicity and CRS

Neurotoxicity and CRS are known to occur independently with distinct timing and kinetics in response to immunotherapy. However the underlying mechanisms of neurotoxicity and CRS appear interlinked. For example, it is known that up to 50% of patients treated with CD19 CAR-T immunotherapy have at least Grade 3 neurotoxicity or CRS symptoms.

Despite the link between neurotoxicity and CRS the anti-IL6 receptor (IL6R) monoclonal antibody tocilizumab which is approved for the prevention and/or treatment CRS, does not also prevent and/or treat neurotoxicity. Tocilizumab is an IL-6 receptor blocker and in severe CRS cases additional steroid based treatment may also be required to prevent death.

Prolonged use of steroids as part of a therapy is known in the art to have many negative side-effects. Depending on the mode of administration, side-effects can include increased appetite, mood changes and difficulty sleeping.

Advantageously, the present invention provides a means for preventing and/or treating neurotoxicity and/or CRS associated with immunotherapy by disrupting the IL-1β-IL-1R1 signalling axis. Advantageously, the present invention also provides a means to treat and/or prevent both neurotoxicity and CRS. In one embodiment, the present invention provides an advantageous means to prevent and/or treat neurotoxicity and/or CRS where additional steroid treatment may not required.

Prevention/Treatment

The term “prevention” in connection with neurotoxicity and/or CRS refers to reducing or eliminating symptoms and/or pathology before they occur.

The term “treatment” in connection with neurotoxicity and/or CRS refers to reducing or eliminating symptoms and/or pathology after symptom onset. The term “treatment” may further include prevention (prophylaxis) of neurotoxicity and/or CRS, or amelioration or elimination of the disorder once it has been established.

As defined herein, the terms “disorder” and “disease” are used synonymously.

As defined herein, the term “subject” refers to a human. It is not intended that the term “subject” be limited to a particular age or sex. Thus, adult and newborn subjects, as well as fetuses, whether male or female, are encompassed by the term.

In one embodiment, the subject is an adult subject. The term “adult” refers to a subject that has reached biological maturity. In another embodiment, the subject is a pediatric subject. The term “pediatric” refers to an infant, child or adolescent.

Administration

As used herein, the term “administering” or “administration” in relation to an IL-1β inhibitor refers to application or delivery of the IL-1β inhibitor to the subject by any route of delivery.

The IL-1β inhibitor of the present invention is administered to the subject using means and methods that are known and standard practise in the art.

Suitably, the IL-1β inhibitor may be administered to a subject by a subcutaneous, intravenous, parenteral or enteral route. Suitably, the administration may be systemic or local.

The term “parenteral” refers to any route in the body other than the mouth and alimentary canal. Parenteral routes are well known in the art and include but are not limited to subcutaneous, intravenous and intramuscular routes.

The term “enteral” refers to any route that involves the gastrointestinal tract. Enteral routes are well known in the art and include but are not limited to oral, sublingual and rectal routes. Suitably, the mode of administration is chosen to be appropriate for the age and symptoms of the subject.

In one embodiment, canakinumab is administered parenterally, preferably intravenously, more preferably subcutaneously. Suitably canakinumab is administered in a liquid formulation.

In one embodiment, anakinra is administered parenterally, preferably intravenously, more preferably subcutaneously. Suitably anakinra is administered in a liquid formulation.

In one embodiment, rilonacept is administered parenterally, preferably intravenously, more preferably subcutaneously. Suitably rilonacept is administered in a liquid formulation.

The IL-1β inhibitor of the present invention is administered in a dose that prevents and/or treats neurotoxicity and/or CRS. Suitably, the dose will be sufficient to reduce or eliminate IL-1β transduction.

A person of ordinary skill in the art can easily determine an appropriate dose of the IL-1β inhibitor according to the present invention, to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

When used in the presently claimed invention, the IL-1β inhibitor canakinumab is preferably administered in a dose of from about 25, 75, 80, 100, 125, 150, 175, 200, 225, 250, 275, 300 mg. Suitably, the dose administered will be adjusted according to the subject's body weight. Suitably, canakinumab may be administered at about 150 mg for patients with a body weight greater than 40 kg. Suitably, canakinumab may be administered at a dose may be about 2 mg/kg or 3 mg/kg when the subject bodyweight is greater or equal to 15 kg, or less than or equal to 40 g. Suitably, canakinumab may be administered 8 weeks before or after further IL-1β inhibitor administration or immunotherapy administration.

In one embodiment, canakinumab may be administered at a dose of about 2 or 4 mg/kg to subjects with a body weight less than or equal to 40 kg. Canakinumab may be administered at a dose of about 150 or 300 mg to subjects with a body weight greater than 40 kg. Suitably, canakinumab may be administered at an interval of 4 weeks before or after further IL-1β inhibitor administration or immunotherapy administration.

In one embodiment, canakinumab is administered at a dose of about 4 mg/kg (with a maximum of 300 mg) to a subject with a body weight greater than or equal to 7.5 kg. Suitably, canakinumab may be administered at an interval of 4 weeks before or after further IL-1β inhibitor administration or immunotherapy administration.

In one embodiment, canakinumab is administered from about 0.05 to 10 mg/kg, preferably from about 0.1 to 5 mg/kg.

Canakinumab is an FDA approved drug and its dosage and administration for a large variety of conditions are well known.

When used according to the invention, the IL-1β inhibitor anakinra is preferably administered in a dose of from about 0.1 to 100 mg/kg per day, preferably about 0.1 to 1 mg/kg per day. A preferred dosage for the treatment of IL-1β mediated disorders may produce blood anakinra concentrations between about 1 and 1000 ng/ml. Suitably, the pharmaceutical formulation comprising anakinra according to the invention preferably comprises anakinra in an amount between about 100 and 200 mg/ml, such as about 150 mg/ml.

In one embodiment, it is preferred that, initially, doses are administered to bring the circulating levels of anakinra above about 5 ng per ml of plasma. Suitably, anakinra is administered at a dose and frequency to achieve continuous saturation of the IL-1R1.

Anakinra is an FDA approved drug and its dosage and administration for a large variety of conditions are well known.

When used according to the invention, the IL-1β inhibitor rilonacept is preferably administered in a dose of about 320 mg. Suitably, the dose may be administered as two administration events of about 160 mg each on the same day. Suitably, rilonacept may additionally be administered at a dose of about 160 mg, preferably weekly. Suitably, rilonacept is administered at a dose of about 4.4 mg/kg up to a maximum of 320 mg for paediatric patients. Suitably rilonacept is administered at a dose of about 2.2 mg/kg up to 160 mg. In one embodiment, rilonacept is not administered more than once weekly.

It is known in the art that Rilonacept is administered weekly by subcutaneous injection to treat Cryopyrin-Associated Periodic Syndromes (CAPS). Rilonacept is an FDA approved drug and its dosage and administration are well known.

The present invention provides compositions comprising IL-1β for use in the prevention and/or treatment of neurotoxicity and/or CRS in a subject undergoing immunotherapy, wherein the IL-1β inhibitor is administered before immunotherapy.

It should be understood that the compositions comprising IL-1β for use according to the invention encompass all embodiments as described herein.

Even though the compositions of the present invention (including their pharmaceutically acceptable salts) can be administered alone, they will generally be administered in admixture with a pharmaceutical carrier, excipient or diluent, particularly for human therapy. Such formulations, along with methods for their preparation, will be familiar to a person of ordinary skill in the art.

The IL-1β inhibitor is administered to the subject as a composition, preferably a pharmaceutical composition. When the IL-1β inhibitor is administered as a pharmaceutical composition, said composition may contain pharmaceutically acceptable carriers or additives, depending on the route of administration. The form of the pharmaceutical composition is well known in the art and readily determinable.

The composition for use according to the invention may be in a lyophilized form. Suitably, the composition for use may be dissolved in an aqueous carrier. Suitably, the composition for use may further comprise any components that promote stability of the IL-1β inhibitor in solution.

The present invention also includes solvate forms of the compositions comprising IL-1β inhibitors.

Suitably, the pharmaceutical composition may comprise buffering agents, tonicity agents, antioxidants, stabilizers, surfactants, bulking agents, chelating agents and preservatives. In one embodiment, the pharmaceutical composition is Kineret®. In one embodiment, the pharmaceutical composition is Ilaris®. In one embodiment, the pharmaceutical composition is Arcalyst®.

In one embodiment, the pharmaceutical composition for use according to the present invention comprises an IL-1β inhibitor and further comprises at least one further active pharmaceutical ingredient (API).

Method of Treatment

In another aspect, the invention provides a method of treating and/or preventing neurotoxicity and/or CRS in a human subject undergoing immunotherapy, the method comprising administering to a human subject in need thereof an IL-1β inhibitor before immunotherapy.

Suitably the method of treatment and/or prevention of neurotoxicity and/or CRS comprises the administration of an IL-1β inhibitor according to the embodiments as described herein. Suitably the subject is undergoing immunotherapy according to the embodiments as described herein.

Suitably, the method for preventing neurotoxicity and/or CRS relates to the prophylactic use of the IL-1β inhibitor. In this respect, the IL-1β inhibitor may be administered to a subject who has not yet contracted the disorder and/or who is not showing any symptoms of the disorder, and/or who has not yet undergone immunotherapy. The subject may have a predisposition for, or be thought to be at risk of developing a disorder and/or may be a candidate for immunotherapy.

The methods for preventing and/or treating a disorder provided by the present invention, may involve monitoring the progression of the disorder and/or the progression of response to immunotherapy.

“Monitoring the progression of the disorder” means to assess the symptoms associated with the disorder and/or immunotherapy treatment over time to determine if they are reducing/improving or increasing/worsening.

Suitably, the dose of the IL-1β inhibitor administered to a subject, or the frequency of administration, may be altered in order to provide an acceptable level of both disorder progression and immunotherapy treatment according to the methods of the invention. The specific level of disorder progression and toxic activities determined to be ‘acceptable’ will vary according to the specific circumstances and should be assessed on such a basis.

In one embodiment, the invention provides the use of an IL-1β inhibitor for the manufacture of a medicament for the treatment and/or prevention of neurotoxicity and/or CRS in a human subject undergoing immunotherapy, wherein the medicament is administered before immunotherapy.

Suitably the treatment and/or prevention of neurotoxicity and/or CRS comprises administering an IL-1β inhibitor according to the embodiments as described herein. Suitably the subject is undergoing immunotherapy according to the embodiments as described herein.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### EXAMPLES

### Example 1: Cytokine Release Syndrome (CRS) Mouse Model

3×10\hat{ }6 Raji tumour cells from ATCC are injected intraperitoneally into a 6-8 week old female SCID-beige mouse (Taconic) and allow to grow for 20 days, until they eventually grow into vascularised solid tumour masses. 30×10\hat{ }6 CD19 CAR T cells are then injected into the mouse. A panel of known CRS markers are monitored e.g. percentage weight change over 65 hours, serum cytokine profile including IL6 levels and/or IL15 levels and/or iNOS⁺ cells. CRS is confirmed in mice displaying, for example, more than 10% weight drop in 65 hours and/or raised IL6 levels. For a more severe CRS model, mCD40L is constitutively expressed with the CD19 CAR T cells (CAR-mCD40L).

### Example 2: CRS in Immunotherapy Following Pre-Administration of IL-1B Inhibitor

The CRS markers are measured as described in Example 1 in tumour-bearing mice which receive anakinra (30 mg/kg mouse weight per day) daily from 10 days to 1 day (day−10 to day−1) before CAR T cell infusion. CAR T cells are injected into mice 7 days after the first T cell activation.

The marker levels measured are compared CRS markers levels with tumour-bearing mice which receive anakinra only during (day 0) or post CAR T cell therapy (day+1 to day+4). Control mice receive PBS supplemented with 2% human serum.

Mice which received pre-administration of anakinra showed significantly less weight loss over a 65 hour period following CAR T cell infusion compared with mice which received anakinra only during or post CAR T cell infusion.

### Example 3: CRS in Immunotherapy Following Administration of IL-1B Inhibitor (Pre, During and/or Post-Administration of Immunotherapy)

The pre-administration protocol described in Example 2 is repeated (day−10 to day−1). The administration of anakinra (30 mg/kg mouse weight per day) is continued on day 0 and/or 1 day to 4 days post CAR T cell infusion (day−10 to day0/or day−10 to day+4).

The marker levels are then measured and compared with levels in tumour bearing mice which receive anakinra only during (day 0) and/or only post CAR T cell therapy (day+1 to day+4). Control mice receive PBS supplemented with 2% human serum.

Mice which received pre-administration dosages of anakinra showed significantly less weight loss over a 65-hour period following CAR T cell infusion compared with mice which received anakinra only during or only after CAR T cell infusion.

1-26. (canceled)

27. A method of prevention and/or treatment of neurotoxicity and/or cytokine release syndrome in a human subject undergoing immunotherapy, the method comprising administering to a human subject in need thereof an IL-1β inhibitor before immunotherapy.

28. The method according to claim 27, wherein the IL-1β inhibitor is administered more than once.

29. The method according to claim 27, wherein the IL-1β inhibitor is administered about 30 days or less before immunotherapy administration.

30. The method according to claim 29, wherein the IL-1β inhibitor is administered 1 to 30 days, 5 to 30 days, 10 to 30 days, 15 to 30 days, 20 to 30 days, or 25 to 30 days before immunotherapy administration.

31. The method according to claim 29, wherein the IL-1β inhibitor is administered 1 to 5 days, 5 to 10 days, 10 to 15 days, 15 to 20 days, 20 to 25 days, and/or 25 to 30 days before immunotherapy administration.

32. The method according to claim 29, wherein the IL-1β inhibitor is administered 30 days or less, 25 days or less, 20 days or less, 15 days or less, 10 days or less, 5 days or less, 4 days or less, 3 days or less, 2 days or less, or 1 day before immunotherapy administration.

33. The method according to claim 27, wherein the IL-1β inhibitor is further administered during immunotherapy administration and/or after immunotherapy administration.

34. The method according to claim 27, wherein the IL-1β inhibitor blocks or sterically hinders the binding of IL-1β to interleukin-1 receptor 1 (IL-1R1).

35. The method according to claim 27, wherein the IL-1β inhibitor is an antibody or antigen-binding fragment thereof, a peptide or a small molecule.

36. The method according to claim 27, wherein the IL-1β inhibitor is an anti-IL1β antibody or antigen-binding fragment thereof.

37. The method according to claim 36, wherein the anti-IL1β antibody is canakinumab or an antigen-binding fragment thereof.

38. The method according to claim 27, wherein the IL-1β inhibitor is an IL-1R1 peptide antagonist.

39. The method according to claim 38, wherein the IL-1R1 peptideantagonist is anakinra.

40. The method according to claim 27, wherein the subject is a human subject.

41. The method according to claim 27, wherein the subject is undergoing immunotherapy for cancer.

42. The method according to claim 27, wherein the prevention and/or treatment of neurotoxicity and/or cytokine release syndrome in a subject is related to a disorder being targeted by T-cell mediated immunotherapy.

43. The method according to claim 27, wherein the immunotherapy is CAR T cell therapy or transgenic TCR T cell therapy, BiTE therapy or an anti-CD3 antibody therapy.

44. The method according to claim 43, wherein the immunotherapy is CAR T cell therapy.

45. The method according to claim 27, wherein the subject is an adult.

46. The method according to claim 27, wherein the subject is a pediatric subject.


SEQUENCE LISTING (5 sequences)

SEQ ID NO: 1 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 2 - protein, 111 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNE

SEQ ID NO: 3 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 4 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 5 - protein, 880 aa, Artificial Sequence
  illustrative sequence for rilonacept
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK